(19) United States
(12) Patent Application Publication
(10) Pub. No.: US 2007/0272121 A1
(43) Pub. Date: Nov. 29, 2007

(54) Ready Mix Flavored Film Coating Systems

(75) Inventors: Suresh Pareek, Mumbai (IN); Ashok Mohanty, Mumbai (IN)

Correspondence Address: HAMRE, SCHUMANN, MUELLER & LARSON, P.C., P.O. BOX 2902, MINNEAPOLIS, MN, 55402-0902, US

(73) Assignee: IDEAL CURES PVT. LTD., Mumbai (IN)

(21) Appl. No.: 10/590,565

(22) PCT Filed: Feb. 28, 2004
(86) PCT No.: PCT/IB04/00515

§ 371 Date: Jun. 11, 2007

(51) Int. Cl. A61K 9/36

(52) U.S. Cl. 106/168.01; 106/172.1

(57) ABSTRACT

The invention comprises a preparation of a ready mix flavored composition, which when reconstituted with water/solvent gives film coatings that are flavored, so that the thin film coating on solid oral dosage forms containing unpleasant tasting ingredients, masks the bitter taste of the medicaments in the oral solid dosage forms. The flavored coating of the invention is comprised of a film—forming substances such as a hydroxy propyl methylcellulose and other polymers and a flavoring agent.

### FIELD OF INVENTION

This invention relates to a ready mix flavored composition for film coating of pharmaceutical oral solid dosage form and to methods of its preparation. In particular, the invention relates to a ready to use flavored film forming composition, easy to reconstitute and capable of forming a flavored film coat for masking the taste of tablets containing bitter principles.

### DESCRIPTION OF PRIOR ART

Film coating of pharmaceutical dosage forms have been developed and are summarized as early as in 1982 in Pharmaceutical Dosage Forms: Tablets, Volume 3 (Eds. Liberman and Lachman, 1982, Marcel Dekker). The reasons for coating may be various and include protection of unstable compositions, protection in the stomach with enteric coating, improving the appearance of the tablet, separation of the ingredients of a tablet if they are incompatible, and masking objectionable odors and taste.

The techniques include aqueous film coating, delayed release coating, granule coating, sugar coating, solvent film coating etc. The thin film coating of pharmaceutical tablets allows efficient, controlled, uniform and reproducible coats. Use of multiple layers of coating, such as the polymeric undercoat/seal coat, polymeric pigmented second coat and polymeric finish coat allows the preparation of very smooth glossy tablets (Ohno, U.S. Pat. No. 4,001,390). This patent and all other cited patents are incorporated by reference herein.

Aqueous thin film coating systems are environmentally more safe compared to non-aqueous systems which involve use of organic solvents in film coating solutions. Thin coatings usually do not alter the disintegration time or dissolution profile characteristics of the tablet. In case of active pharmaceutical ingredients which have a strong and objectionable bitter taste a thick or insoluble coating may be required to mask the taste and odor which in turn may affect the disintegration and dissolution characteristics of the dosage form. Powdery tablets have been coated (John et al.; U.S. Pat. No. 4,302,440) to retard their dissolution. Non aqueous polymeric wet granulation (Gans et al., U.S. Pat. No. 3,388,041) or enteric coats (Jeffries, U.S. Pat. No. 3,149,040) have also been used. Singiser, U.S. Pat. No. 3,256,111 and Brindamour, U.S. Pat. No. 3,383,236 discuss non-aqueous film-coating systems designed to be applied to a variety of tablets containing different active ingredients.

A film coat is a thin coat wherein the weight in increase of tablet weight is around 2 to 3% of the average weight. Attempts to mask taste and odor of active pharmaceutical ingredients in tablets have resulted in thick coatings, slow dissolving coatings and sugar coatings (coatings containing sweeteners). The disadvantage of thick unflavored coatings include partial dissolution of coating in the mouth (for persons who have difficulty in swallowing) thus decreasing its effectiveness to masking the odor and taste.

Sugar coating to improve palatability and appearance are also known However it increases the tablet weight and size and is very time consuming as the process involves application of a hydrophobic layer, sub-coat, sugarcoat, coloring and polishing. In addition, they do not include flavors to disguise bitter taste.

Flavorings have traditionally been used in liquid medicines to mask the bitter taste and odor For tablets usually the bitter taste is masked by coating it with a waxy material or water soluble high molecular weight polymer without any flavoring agent. The inert film coat thus does not expose the bitter ingredient to the taste receptors in the mouth and thus functions in masking. However if the residence time is more in the mouth of an individual, then the thin coating may partially dissolve and the individual may feel the bitter taste.

Thus it will be an added advantage if flavor is present in the coat which helps to avoid the bitter taste in case the non flavored film coat dissolves quickly in the mouth of the individual.

It is believed that the previous uses of flavorings or fragrances in readymix film coatings for pharmaceutical tablets have utilized flavors in an organic solvent coating for masking objectionable odor as referenced in Motoyama, U.S. Pat. No. 4,154,636, Singiser, U.S. Pat. No. 3,256,111 and John, U.S. Pat. No. 4,302,440.

A major functional advantage of tablet coatings has been to aid in tablet identification. Thus, the use of coatings containing pigments on tablets provides a way to identify tablets by color. Pigment addition also allows the tablets to have a more uniform and pleasing appearance. Tablet coatings comprising a colored film coating have been prepared, for example, by dispersing an anhydrous pigment suspension in a polymer solution (Signorino U.S. Pat. No. 3,981,984). However, persons with impaired vision often have difficulty in being sure that they are taking the correct medicine even with color-coded tablets, which is solved by flavored tablets.

### OBJECTIVE OF THE INVENTION

This invention provides an unexpected advantage of masking unpleasant medicinal tastes such as that associated with bitter tasting medicinal compositions by providing a readymix composition that can be reconstituted in water/organic solvents to provide a flavored film-coating system to be applied to tablets. By the use of a dry blend composition consisting of a polymer or a mixture of polymers and a flavoring agent, which can be reconstituted in water, there is obtained a ready to use system for application to bitter tasting medicaments to mask their taste.

Another object of the invention is to provide a ready mix composition for film coating that does not contain any sweetening agent. This is specially important for the diabetic population who prefer to avoid sweetening agents in any form.

Another object of the invention is to provide a ready mix composition for film coating that will enable oral identification of the tablet due to the particular flavor of the coat being associated with the particular core tablet composition. Oral flavor identification of this invention allows visually impaired as well as other persons to know that the correct medication is being taken so that mistakes in medication may be avoided.

Another object of the invention is to provide a ready mix composition for film coating of tablets that enables different strengths of the same active ingredient, such as a prescription medicine, to be identified by different flavored coatings being applied to the different ingredient strengths.

Another object of the invention is to provide a ready mix composition for film coating that enables increased compliance with prescribed medicine schedules. The flavored coat provides a flavored oral stimulus that enables those who have taken flavor-coated tablets to have an enhanced memory of having taken the tablet through remembrance of the particular flavor of coating. The flavored coating of the invention also enhances the appeal of a particular medicine so that persons do not avoid taking their medicine.

Another object of the invention is to provide ready mix composition for film coating by forming a film-coating suspension, so that the tablets prepared from the same are smooth easily swallowed tablet and to facilitate swallowing ease through increased salivation if the coated tablet lingers in the mouth and is tasted.

Another object of the invention is to provide ready mix for preparation of the film coating suspension, so that the tablets prepared from the same does not slow the dissolution of the core tablet and in which the bioavailability of the active ingredients is not significantly reduced or impaired.

Another object of the invention is to provide a ready mix composition for film coating process for preparing a flavor-coated pharmaceutical tablet, which decreases the process time and increases productivity.

### SUMMARY OF THE INVENTION

The invention relates to a ready mix dry blend composition for preparation of a film coating suspension, to be used for coating of tablets containing unpleasant tasting active ingredients such as albendazole, multivitamins and nutritional supplements. The method of the invention comprises reconstitution of a ready mix dry blend composition to give a coating suspension for application on tablets so as to give film coating. The ready mix dry blend comprises a water-soluble hydrophilic polymer or water insoluble hydrophobic polymer or a mixture of water-soluble hydrophilic polymer and water insoluble hydrophobic polymer and a flavoring agent. The water soluble hydrophilic polymer preferred is hydroxypropyl methyl cellulose whereas the water insoluble hydrophobic polymer preferred is ethylcellulose. There is no sweetening agent added to the dry mix composition as the flavoring agent in addition to masking the odor also masks the taste and thus no extra sweetening agent is required.

### DESCRIPTION OF THE PREFERRED EMBODIMENT

The method of this invention comprises standard pharmaceutical coating techniques and conditions using a flavored coating. In particular, pharmaceutical core tablets are continuously (i.e., not intermittently) spray-coated with a thin film coating containing a flavoring agent. Suitably formulated core tablets are placed in a coating chamber. A preferred composition of ready mix coating material, of an excessive volume to allow coating losses to the pan, exhaust and spray equipment, is sprayed into the coating chamber until the coated tablets show a weight increase of 2 to 5.0 parts per 100 parts by weight of the core tablet weight. The preferred method of the invention comprises a step of reconstituting the readymix composition in water and/or organic solvents and a one-step continuous spray-coating process to apply the thin flavored coating. Thus, the preferred embodiment is distinguishable from sugar-coating processes in which multiple layers of sugar-containing coating are applied, each followed by a drying period. It is also possible to apply more than one flavored coat or to apply the flavored coating after an initial sealing coat. If any coating, such as a wax coating, is applied after the flavored coating, it must be designed to allow taste perception of the flavored coating.

The preferred pharmaceutical tablet with which the flavored ready mix coating of this invention is used contains albendazole. These tablets contain 100 mg of the active ingredient, in a typical tablet of weight 650 mg. The coating increases the weight of the tablets by an average of 4%.

The advantages of this invention are also realized through flavor-coating of other bitter or objectionably strong flavored tablets, especially those that bleed through the thin coating. Such other bitter or objectionable-tasting active ingredients include, but are not limited to chloroquine phosphate, quinine sulphate, roxithromycin, clarithromycin, cephalosporins, ampicillin and cloxacillin trimethoprim, sulfamethoxazole, guaifenesin, bupropion, chlorpheniramine maleate, dextromethorphan, azidothymidine and other salts or combinations of these ingredients and those of the preferred embodiment. The invention may also be used with sustained-release formulations.

The preferred ready mix composition for film coating of this invention is comprised of a commercial film-coating product designed for aqueous/non aqueous film coating containing water soluble hydrophilic film-forming polymers like, hydroxypropyl methylcellulose polyvinyl pyrrolidone, methacrylic acid polymers, carragenan, sodium alginate, plasticizers such as polyethylene glycol, propylene glycol, soyalecithin, dibutyl phthalate, diethyl phthalate and glycerin and optionally containing titanium dioxide (or other colorant or opacifying agent) in combination with a suitable flavoring agent but without a sweetening agent.

Other preferred embodiments of this invention also include ready mixes comprised of a commercial film-coating product designed for aqueous/non aqueous film coating containing water insoluble hydrophobic film-forming polymers like, ethylcellulose, plasticizers such as polyethylene glycol, propylene glycol, soyalecithin, dibutyl phthalate, diethyl phthalate and glycerin and optionally containing titanium dioxide (or other colorant or opacifying agent) in combination with a suitable flavoring agent but without a sweetening agent.

Another embodiment of this invention also include ready mixes comprised of a commercial film-coating product designed for aqueous/non aqueous film coating containing a mixture of a water soluble hydrophilic polymer like methylcellulose and a water insoluble hydrophobic film-forming polymers like, ethylcellulose, plasticizers such as polyethylene glycol, propylene glycol, soyalecithin, dibutyl phthalate, diethyl phthalate and glycerin and optionally containing titanium dioxide (or other colorant or opacifying agent) in combination with a suitable flavoring agent but without a sweetening agent.

A suitable blend comprises 0 to about 20% w/w titanium dioxide or colorant, about 5 to about 95% w/w hydroxypropyl methylcellulose, and 0 to about 25% w/w polyethylene glycol. The most preferred embodiment comprises the polymer hydroxy propyl methyl cellulose 50-65% w/w, titanium dioxide 8-15% w/w, plasticizer 12-25% w/w, non-water additives such as colorants 5-25% w/w and talc 3-5% w/w, along with the preferred flavor.

The ready mix blend is added to purified water or isopropyl dichloromethane mixtures at ambient temperature in a vortex mixer and gelling allowed to take place by stirring for 45 minutes.

Flavorings may be obtained from a variety of sources with the relevant criteria being strength and pleasing nature of the flavor. The flavor agent selected, the film coating dispersion formulation and the amount of solids sprayed on to the tablet affect the flavor strength of the desired product. The preferred flavoring amount is readily determined by balancing the goal of adding an amount sufficient to mask the core tablet taste and provide a distinct, characteristic and pleasing taste, and the goal of keeping the tablet from being too much like a candy or mint product. The desired strength of the flavoring may vary depending on the type of tablet and the intended recipients and the identity of the flavoring.

The following equipment was used in practicing the method of this invention as demonstrated in the examples. The coating pan was an 8-inch perforated coating pan rotating at about 20 rpm and providing about 1000 cu ft/min of inlet air at a temperature of 70.degree. C. Tablet bed temperature was maintained at 45 degree. C. Although 45 degree. C. is the optimum temperature, acceptable quality coatings may be obtained at tablet temperatures from 38.degree.-55.degree. C. The spraying unit was an air-atomized Bullows 630 gun, supplying the coating suspension through peristaltic pump.

Equipment to be used for scale-up operations would be obvious to a person skilled in the art of pharmaceutical coatings. For example, larger pans of 48-inches would accommodate increased number of core tablets. It is also clear that the inlet air volume, rotation speed of the pan and temperature are interactive factors in coating operations and the cited parameters and equipment are for illustration purposes only and do not limit the invention. Although use of air spraying units results in more even coating of core tablets due to better droplet-size control, airless spraying units may also be utilized.

When the flavor-coated tablets as prepared by the method of this invention are administered to a recipient, the positive taste perception of the flavored coat of the invention lasts on the tongue for at least five seconds, which is generally more than enough time for the tablet to be swallowed before the tablet's bitterness becomes objectionable.

Because the flavors used in this invention are volatile, it would be expected that the high temperatures employed during manufacturing would cause the flavoring agents to volatilize during the spray-coating process and the flavors to be lost. The surprising and unexpected result in the actual practice of this invention is that when the flavoring agents are incorporated into the coating dispersion, the flavors is retained.

The following examples illustrate the invention without limiting it to the examples. In particular, numerous strongly flavored agents, such as other fruit flavors, other mint-related flavors and other natural and artificial flavors, may be employed in lieu of those in the examples.

### EXAMPLE 1

Core tablets of antihelmintic drug Albendazole 100 mg were compressed at tablet weight of 640 mg in a capsule shaped punch of 12 mm×6 mm size. The hardness and friability of the tablets were with the pharmacopoeial limits. Different dry blends of the ready mix were prepared and used for the coating the core tablets, using a 8 inch coating pan. The spray system employed was a bullows atomized air gun supplied with the coating solution through the peristaltic pump.

A ready mix coating dispersion formulation, was prepared by dry blending the ingredients in a rapid mixer granulator, with the following percentages (w/w):

| | Specific | Ready Mix | Ready Mix | Ready Mix |
|---|---|---|---|---|
| | ingredient | Composition A | Composition B | Composition C |
| Ingredients | used | (% w/w) | (% w/w) | (% w/w) |
| Polymer | HPMC | 45% | 55% | 60% |
| Colorant | Titanium | 20% | 27% | 20% |
| | dioxide | | | |
| Plasticizer | Polyethylene | PEG 400 15% | PEG6000 11% | PEG 6000 12% |
| | glycol | | | |
| Non-soluble | Talc | TALC 18% | AEROSIL 3% | TALC 3% |
| additives | | | | |
| Flavor | Chocolate | 2% | 4% | 5% |

The best film coated formulation was chosen out of the above formulations after taste perception studies and found that the formula C gave a good film coat coupled with a good dissolution of the film flavor on the tongue.

### EXAMPLE 2

Core tablets of MULTIVITAMINS were compressed at tablet weight of 940 mg in a capsule shaped punch of 18 mm×8.5 mm size. The hardness and friability of the tablets were with the pharmacopoeial limits. Different dry blends of the ready mix were prepared and used for the coating the core tablets, using a 8-inch coating pan. The spray system employed was a bullows atomized air gun supplied with the coating solution through the peristaltic pump.

| | Specific | Ready Mix | Ready Mix | Ready Mix |
|---|---|---|---|---|
| | Ingredient | Composition A | Composition B | Composition C |
| Ingredients | used | (% w/w) | (% w/w) | (% w/w) |
| Polymer | HPMC | 40% | 45% | 50% |
| | Ethylcellulose | 10% | 10% | 9% |
| Colorant | Titanium dioxide | 26% | 24% | 20% |
| Plasticizer | Polyethylene | PEG 400 12% | PEG 400 13% | PEG 6000 12% |
| | glycol | | | |
| Non-soluble | Talc/Aerosil | Talc 10% | Aerosil 5% | Talc 4% |
| additives | | | | |
| Flavor | Vanilla | 2% | 3% | 5% |

### EXAMPLE 3

Core tablets of a Nutritional supplement containing organic calcium and vitamin D3 were compressed at tablet weight of 1.5 gm in a capsule shaped punch of 19 mm×9.5 mm size. The hardness and friability of the tablets were with the pharmacopoeial limits. Different dry blends of the ready mix were prepared and used for the coating the core tablets, using a 8-inch coating pan. The spray system employed was a bullows atomized air gun supplied with the coating solution through the peristaltic pump.

| | Specific | Ready Mix | Ready Mix | Ready Mix |
|---|---|---|---|---|
| | Ingredient | Composition A | Composition B | Composition C |
| Ingredients | used | (% w/w) | (% w/w) | (% w/w) |
| Polymer | RD100 | 50% | 55% | 60% |
| Colorant | Titanium | 27% | 27% | 15% |
| | dioxide and | | | |
| | color lake | | | |
| Plasticizer | Polyethylene | PEG 400 12% | PG 11% | PEG 6000 12% |
| | glycol | | | |
| Non-soluble | Talc | TALC 10% | AEROSIL 5% | TALC 5% |
| additives | | | | |
| Flavor | Vanilla | 1% | 2% | 8% |

### EXAMPLE 4

Core tablets of a Multivitamin formulation were compressed at tablet weight of 1.3 gm in a capsule shaped punch of 16 mm×8 mm size. The hardness and friability of the tablets were with the pharmacopoeial limits. Different dry blends of the ready mix were prepared and used for the coating the core tablets, using a 8-inch coating pan. The spray system employed was a bullows atomized air gun supplied with the coating solution through the peristaltic pump.

| | Specific | Ready Mix | Ready Mix | Ready Mix |
|---|---|---|---|---|
| | Ingredient | Composition A | Composition B | Composition C |
| Ingredients | used | (% w/w) | (% w/w) | (% w/w) |
| Polymer | Polyvinyl | 45% | 50% | 60% |
| | alcohol | | | |
| Colorant | Titanium | 15% | 29% | 13% |
| | dioxide and | | | |
| | color | | | |
| Plasticizer | Soya lecithin | 15% | 10% | 10% |
| Non-soluble | Talc/Aerosil | Talc 15% | Aerosil 5% | Talc 10% |
| additives | | | | |
| Flavor | Ethyl vanillin | 5% | 6% | 7% |

We claim:

1. A ready mix flavored composition for film coating of pharmaceutical oral solid dosage form comprising of a polymer and a flavoring agent but without a sweetening agent and capable of masking the unpleasant taste of a solid core.

2. A ready mix flavored composition of claim 1, wherein the polymer is a water soluble hydrophilic polymer preferably hydroxypropyl methyl cellulose.

3. A ready mix flavored composition of claim 1, wherein the polymer is a water insoluble hydrophobic polymer preferably ethylcellulose.

4. A ready mix flavored composition of claim 1, wherein there may be a combination of a water-soluble hydrophilic polymer preferably hydroxypropyl methyl cellulose and a water insoluble hydrophobic polymer preferably ethylcellulose.

5. A ready mix flavored composition of claim 1, containing a flavoring agent that may be volatile or non-volatile.

6. A ready mix flavored composition of claim 1, further containing a plasticizer, non-soluble additives and a colorant.

7. A ready mix flavored composition of claim 1, wherein the polymer comprises from about 10-80% w/w based on the total weight of the composition.

8. A ready mix flavored composition of claim 1, wherein the flavoring agent comprises from about 0.1-25% w/w based on the total weight of the composition.

9. A ready mix flavored composition of claim 1, which further contains a plasticizer from about 5-25% w/w, non-soluble additives such as colorants, detackifiers and opacifying agents from about 5-25% w/w based on the total weight of the composition.

10. A process for manufacture of a flavored ready mix composition comprising a polymer, a flavoring agent, a plasticizer, non-soluble additives and colorants comprising the steps of:
a) blending all components of the composition in a suitable blender or mixer to achieve a uniform mix of the dry blend.
(b) Obtaining the film coating composition by dispersing the ready mix of (a) in water and or organic solvents.
